(19) United States
(12) Patent Application Publication
(10) Pub. No.: US 2008/0140119 A1
(43) Pub. Date: Jun. 12, 2008

(54) Pacifier

(76) Inventors: Azriel Bezalel Machtiger, Herzeliya (IL); Gilead Asseo, Kfar Maas (IL); Ronella Marom, Tel-Aviv (IL); Benjamin Drori, Tel-Aviv (IL)

Correspondence Address: Pearl Cohen Zedek Latzer, LLP, 1500 Broadway, 12th Floor, New York, NY, 10036, US

(21) Appl. No.: 11/637,247

(22) Filed: Dec. 12, 2006

(51) Int. Cl. A61J 17/00; A61B 5/00

(52) U.S. Cl. 606/234

(57) ABSTRACT

A baby pacifier having a base portion and an attached nipple portion, the pacifier being arranged to actuate an alarm if the pacifier has fallen out of the mouth of a baby using same, a detection circuit being substantially disposed in the base portion of the pacifier, the detection circuit including an interconnected heat sensor positioned inside the pacifier nipple, the detection circuit being arranged to generate a first signal on detecting a significant fall in the temperature measured by the heat sensor, an alarm transmission circuit being responsive to the first signal and in response to receipt thereof generating a second signal, the second signal being transmitted to and received by switching means supplying/cutting electric power to an alarm means.

### FIELD OF THE INVENTION

The present invention relates to devices aimed at the reduction of the occurrence of Sudden Infant Death Syndrome (SIDS). More particularly, the invention provides a baby pacifier which issues a warning if fallen out of the mouth of a baby.

### BACKGROUND OF THE INVENTION

SIDS is a most worrying occurrence. The sudden discovery that a healthy baby is dead provides a terrible shock and grief for the parents thereof. Yet this is what has happened in 2,162 cases in the year 2003 in the USA alone. The situation was even worse before the 1990 recommendation was adopted by the public, that babies up to a year old should be laid down to sleep on their back. Recently (October 2005) the US Academy of Pediatrics appointed a task force to see what, if anything, can be done to further reduce occurrences. A new solution remarkable in its simplicity was proposed—“we recommend pacifier use for infants up to 1 year of age” “The pacifier should be offered to the infant when being placed for all sleep episodes, including daytime naps and nighttime sleeps” “This is a US Preventive Services Task Force level B strength of recommendation, based on the consistency of findings and the likelihood that the beneficial effects will outweigh any potential negative effects.” This recommendation appears in an article by Hauck et al. in PEDIATRICS 2005:116, pages 716-723. Their conclusions were based on the investigation of 384 cases of SIDS.

It is obvious that protection of the baby according to this recommendation demands not only the provision of a pacifier but also requires means to ensure that the pacifier is indeed positioned in the mouth of the baby at all sleeping episodes. Parents, attendants and baby-sitters, particularly at night, can however be quite unaware that the baby has fallen asleep without the pacifier, and that the baby is thus in increased danger of SIDS. The present invention is thus directed at finding a workable solution for such a situation.

In U.S. Pat. No. 5,581,238 Chang et al. describe and claim a pacifier with a fever heat alarm. The invention includes a LCD on the pacifier body, a battery housing, an antenna and transmitter for delivery of alarm signals which can be received by an amplifying circuit of a receiver housed in a TV set or a stereo set. A buzzer is housed in the pacifier body, which can be most disturbing to a baby. If the pacifier falls out of the mouth of the baby no alarm is issued because the temperature thereof is below 37.2° C. or whatever maximum has been set.

### SUMMARY OF THE INVENTION

It is therefore one of the objects of the present invention to provide a pacifier which gives warning if said pacifier is no longer in the mouth of a baby being protected thereby.

It is a further object of the present invention to provide such a pacifier which is simple to use and on casual examination appears similar to prior-art pacifiers.

The present invention achieves the above objects by providing a baby pacifier having a base portion and an attached nipple portion, said pacifier being arranged to actuate an alarm if said pacifier has fallen out of the mouth of a baby using same, a detection circuit being substantially disposed in said base portion of said pacifier, said detection circuit including an interconnected heat sensor positioned inside the pacifier nipple, said detection circuit being arranged to generate a signal in response to the detection of a significant fall in the temperature sensed by said heat sensor, and/or the trend of temperature degradation, and/or the rate of temperature degradation. The signal is then filtered which then activates an alarm circuit being responsive to said signal and in response to receipt thereof generating a second signal, said signal being used to operate a device that produces an audio alarm, and or a visual alarm, and or an active or reactive electromagnetic signal.

In another preferred embodiment of the present invention there is provided a baby pacifier wherein said second signal is a radio-frequency signal which is transmitted or effected by a circuit including a broadcast antenna disposed in said base portion of said pacifier, said RF signal being received by said remote switching means through an antenna.

In a further preferred embodiment of the present invention there is provided a baby pacifier wherein said second signal is an infra-red or visual signal which is generated by said alarm transmission circuit signal and is received by said remote switching means disposed in sight of said pacifier.

In a further preferred embodiment of the present invention there is provided a baby pacifier wherein said second signal is an ultra sound or audible signal which is generated by said alarm transmission circuit signal and is received by said remote switching means disposed in sight of said pacifier.

In yet a further preferred embodiment of the present invention there is provided a baby pacifier wherein said remote alarm is connectable to a baby crib.

In another preferred embodiment of the present invention there is provided a baby pacifier wherein said remote alarm is configured for wall suspension.

In yet another preferred embodiment of the present invention there is provided a baby pacifier wherein said remote alarm has an audible output.

In a further preferred embodiment of the present invention there is provided a baby pacifier wherein said remote alarm has a visible output.

In a further preferred embodiment of the present invention there is provided a baby pacifier wherein said base portion caries at least one button cell connected to supply power for operation of the electric/electronic circuits disposed in said base portion of said pacifier.

In a most preferred embodiment of the present invention there is provided a baby pacifier wherein said detection circuit further includes a timing device and calculating means to determine whether the calculated rate of fall of temperature equals or exceeds the set rate of temperature fall to be expected when said pacifier falls out of the mouth of said baby, such rate of temperature change causing said detection circuit to generate said first signal. Preferably the rate of temperature fall causing said detection circuit to generate said first signal is set in the range 0.5-10 degrees centigrade per minute.

The present invention relates to a pacifier which activates an alarm if the pacifier is no longer in the mouth of the baby. This is achieved by measuring the heat of the pacifier nipple in a manner to generate an alarm when temperature drops to about 30-34° C. or below. Furthermore, in preferred embodiments the rate of temperature fall is automatically calculated, and if said rate is above a certain value the alarm is sounded, as fast cooling indicates that the nipple is no longer in the mouth of the baby. Any alarm signal generated may be remote from the pacifier itself to prevent disturbing the baby being monitored.

It will be realized that the novel device of the present invention can be adapted to mass production using up-to-date printed circuit techniques. As the prevention of SIDS is a world-wide problem it is anticipated that even larger quantities will be sold without the need to capture a large slice of the total market. One factor making possible such low cost is that no LCD display is needed. While the pacifier is expected to have a usage life of 4-5 months before requiring replacement, the alarm device remains a one-time expense.

The type of alarm output is mainly a choice for those, usually the parents, who are responsible for the care of the baby. An audible alarm is probably the best general choice, except for the hard of hearing who require a visible alarm. A tactile (vibration) alarm can also be provided for those preferring this method. At a small extra cost a combined alarm can be offered.

The best location for the alarm unit is probably on the wall of the room containing the baby, as this allows infra-red communication. However the alarm unit could be in another room, using radio communication, or clipped to the baby crib being used being convenient for cable communication.

Most importantly, the present inventor has also considered the possibility of the alarm unit being disposed on the pacifier itself.

The implementation of the present invention could be utilized either by analog hardware, digital hardware, or software or a combination of the above.

The invention will now be described further with reference to the accompanying drawings, which represent by example preferred embodiments of the invention. Structural details are shown only as far as necessary for a fundamental understanding thereof. The described examples, together with the drawings, will make apparent to those skilled in the art how further forms of the invention may be realized.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram representing of a preferred embodiment of the pacifier according to the invention;

FIG. 2 is an end view of an alarm unit attached to a baby crib;

FIG. 3 is a rear view of an alarm unit suited for wall attachment;

FIG. 4 is a detail sectional view of a battery housing for the pacifier;

FIG. 5 is an electric diagram of a preferred embodiment configured to operate on the basis of temperature fall under a certain predetermined value;

FIG. 6 is an electric diagram of a most preferred embodiment configured to operate on the basis of rate of temperature fall and/or the trend thereof; and

FIG. 7 is an elevational view of a pacifier wherein the printed circuit board is disposed in a chamber adjacent to the base of the nipple.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

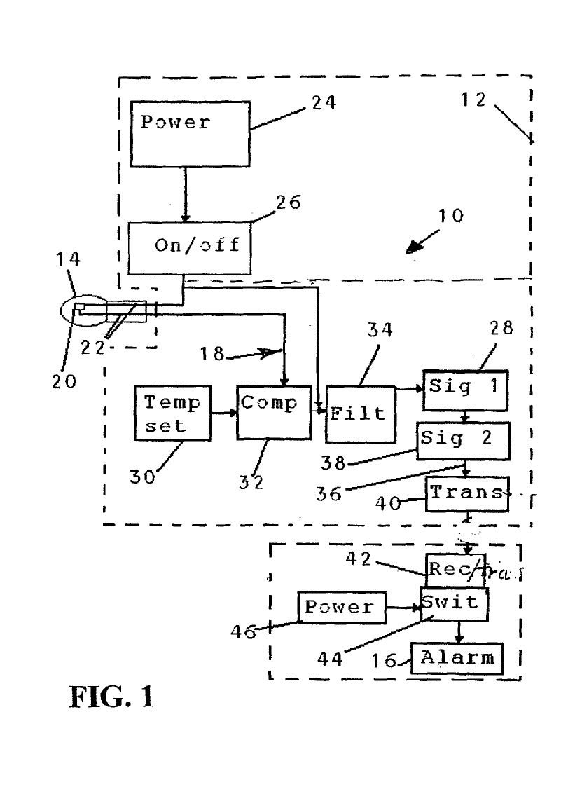

There is seen in FIG. 1 a representation of a baby pacifier 10 having a base portion 12 and an attached nipple portion 14.

The pacifier 10 is arranged to actuate a remote alarm 16 if the pacifier has fallen out of the mouth of a baby using same.

A temperature detection circuit 18 is substantially disposed in the base portion 12 of the pacifier 10. The only part of the detection circuit inside the nipple 14 is a miniature heat sensor 20 and its electric connections 22. Low voltage power 24 is provided and is controlled by an on/off switch 26.

The detection circuit 18 is arranged to generate a first alarm signal 28 on detecting a significant fall in the temperature measured by the heat sensor 20, for example 32° C., a sure sign that the nipple 14 is not in the mouth of the baby. The detected temperature is then passed to a comparator 32 for comparison with the set temperature 30, and if appropriate issues a first signal 28. The detection circuit 18 shown includes a filter 34.

An alarm transmission circuit 36 is responsive to the first signal 28, which is usually too weak to switch on an alarm. Therefore the first signal 28 is arranged to generate a second signal 38, for example by means of an amplifier 40 seen in FIG. 7. The second signal 38 is sufficiently strong for transmission.

The second signal 38 is received 42 by remote switching means 44 supplying electric power 46 to the remote alarm 16. Power for the alarm 16 can be AC line voltage.

Transmission from the pacifier to the alarm is accomplished either by a radio-frequency (RF) signal or by infra-red (IR) devices, or any other method.

The alarm has an audible output, or a visible output or both, or an ultrasound signal adopted to generate an audible sound located at a remote location.

Transmission of the second signal is effected in the present embodiment by sending a RF signal generated by a circuit 36 including a broadcast antenna disposed in the base portion of the pacifier. The receiver 42 of the RF signal can be in a room other than where the baby lies. The RF signal is received by the remote switching means 44 through a receiving antenna.

In a similar embodiment transmission of the second signal 38 is effected by an infra-red (IR) transmitter disposed on the pacifier. An IR receiver 42 operates a remote switching device 44 thus connecting the alarm 16 to its power source 46. The IR receiver 42 is disposed in sight of the (IR) transmitter.

With reference to the rest of the figures, similar reference numerals have been used to identify similar parts.

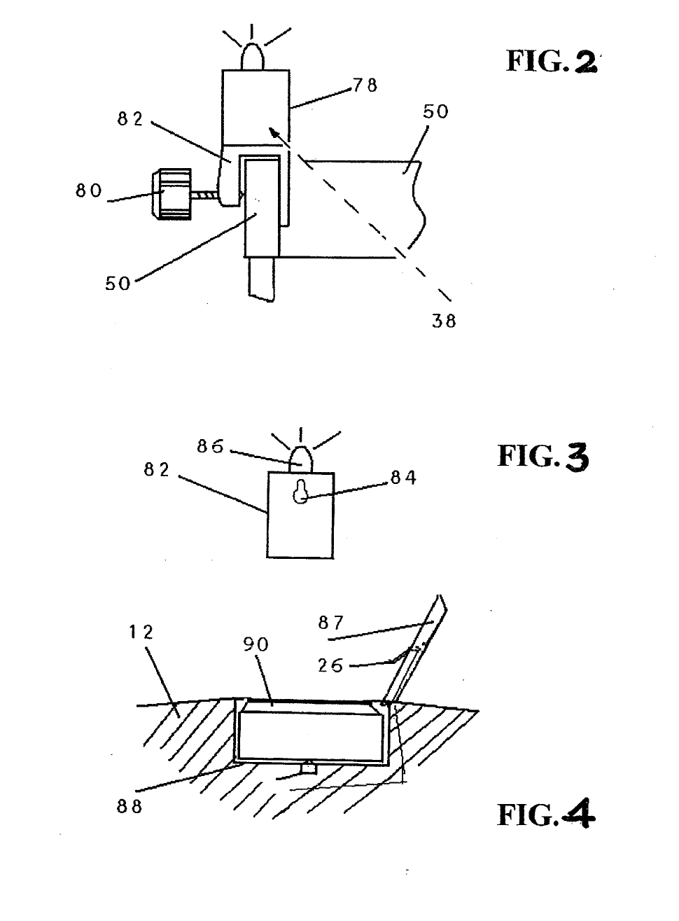

Referring now to FIG. 2, there is depicted a detail of a remote alarm unit 78 responsive to a second signal 38 sent by a baby pacifier. The alarm casing is mechanically connected to the baby crib 50. An inverse U-shaped portion 82 of the alarm casing is held to the upper bar of a baby crib 50 by means of a hand-operated clamp screw 80.

FIG. 3 shows a remote alarm unit 82 configured for wall suspension. A key-hole slot 84 in the rear cover of the alarm allows convenient and removable means for this purpose. For better stability a pair of slots 84 can be provided.

The alarm unit 82 seen is configured to provide a visible output, for example a flashing red light 86.

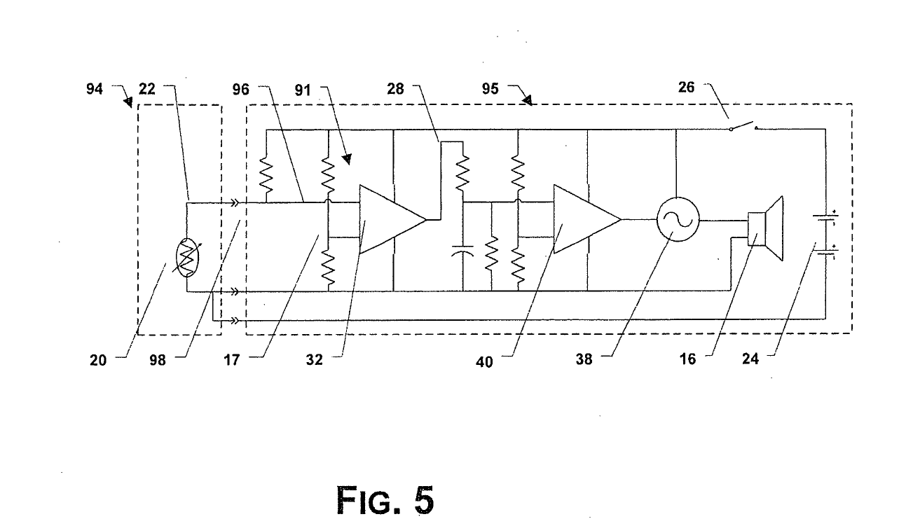

FIG. 4 illustrates the battery compartment 88 of a baby pacifier. The pacifier carries a button cell 90 connected to supply power for operation of all the electric/electronic circuits inside the pacifier. If necessary the battery holder can be configured to hold one cell or more 90 in series. The compartment 88 seen is of the type commonly found in hearing aids.

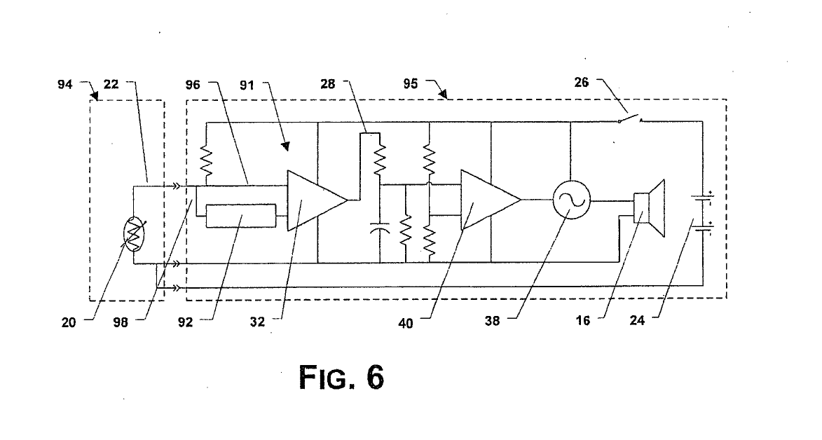

Seen in FIG. 5 is a suitable electronic layout for baby pacifier 94 wherein the detection circuit 91 further includes means to determine the temperature value 17. In the present embodiment comparator 32 is connected by conductors 22, 96 directly to the thermistor 20. The comparator 32 is provided with all the data needed to calculate rate of fall (if any) of temperature. If the rate of temperature fall equals or exceeds the set rate of temperature fall to be expected when the pacifier has fallen out of the mouth of the baby, a first signal 28 is generated.

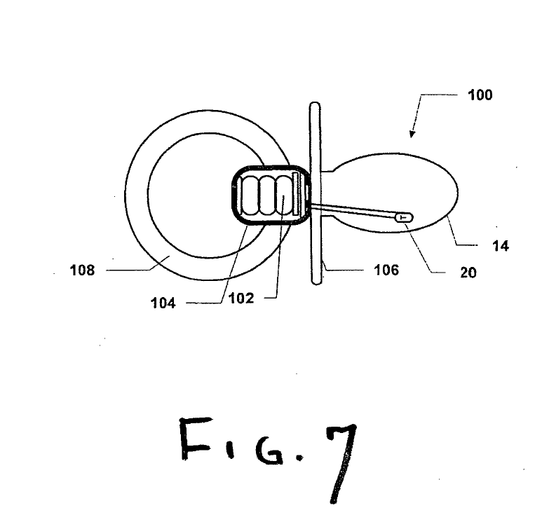

Seen in FIG. 6 is a suitable electronic layout for baby pacifier 94 wherein the detection circuit 91 further includes a time delay device 92. In the present embodiment comparator 32 is connected by conductors 22, 96 directly to the thermistor 20. A further conductor 98 also receives current from the thermistor 20 but this current is first routed through the time delay device 92. Thus the comparator 32 is provided with all the data needed to calculate rate of fall (if any) of temperature. If the rate of temperature fall equals or exceeds the set rate of temperature fall to be expected when the pacifier has fallen out of the mouth of the baby, a first signal 28 is generated.

The figure also shows a number of resistors and a capacitor as is common in electronic circuits.

Typically the rate of temperature fall causing the detection circuit 91 to generate the first signal is set in the range 0.5-10 degrees centigrade per minute.

Turning now to FIG. 7, there is seen an embodiment 100 wherein the printed circuit board (PCB) 102 carrying the detection 18 and alarm transmission circuit 36 previously described is disposed in a chamber 104 positioned adjacent to the pacifier plate 106 on a side opposite to the nipple 14. Also seen in the figure are a retaining ring 108, the outside of the chamber 104 and the thermistor 20 acting as a heat sensor. The thermistor 20 could be replaced by IR detector.

The scope of the described invention is intended to include all embodiments coming within the meaning of the following claims. The foregoing examples illustrate useful forms of the invention, but are not to be considered as limiting its scope, as those skilled in the art will be aware that additional variants and modifications of the invention can readily be formulated without departing from the meaning of the following claims.

1. A baby pacifier having a base portion and an attached nipple portion, said pacifier being arranged to actuate an alarm if said pacifier has fallen out of the mouth of a baby using same, a detection circuit being substantially disposed in said base portion of said pacifier, said detection circuit including an interconnected heat sensor positioned inside the pacifier nipple, said detection circuit being arranged to generate a first signal on detecting a significant fall in the temperature measured by said heat sensor, an alarm transmission circuit being responsive to said first signal and in response to receipt thereof generating a second signal, said second signal being transmitted to and received by switching means supplying/cutting electric power to an alarm means.

2. The baby pacifier as claimed in claim 1, wherein said alarm means being remote of said pacifier.

3. The baby pacifier as claimed in claim 2, wherein an electric cable is arranged to carry said second signal from said alarm transmission circuit to said remote switching means.

4. The baby pacifier as claimed in claim 3, wherein said electric cable is arranged to also supply low voltage power for operation of the electric/electronic circuits disposed in said base portion of said pacifier.

5. The baby pacifier as claimed in claim 1, wherein said second signal is a radio-frequency signal which is transmitted by a circuit including a broadcast antenna disposed in said base portion of said pacifier, said RF signal being received by said switching means through a receiving antenna.

6. The baby pacifier as claimed in claim 1, wherein said second signal is an infra-red signal which is generated by said alarm transmission circuit signal and is received by said switching means disposed in sight of said pacifier.

7. The baby pacifier as claimed in claim 1, wherein said remote alarm is connectable to a baby crib.

8. The baby pacifier as claimed in claim 1, wherein said remote alarm is configured for wall suspension.

9. The baby pacifier as claimed in claim 1, wherein said alarm has an audible output.

10. The baby pacifier as claimed in claim 1, wherein said alarm has a visible output.

11. The baby pacifier as claimed in claim 1, wherein said alarm is ultrasound.

12. The baby pacifier as claimed in claim 1, wherein said base portion carries at least one button cell connected to supply power for operation of the electric/electronic circuits disposed in said base portion of said pacifier.

13. The baby pacifier as claimed in claim 1, wherein said detection circuit further includes a timing device and calculating means to determine whether the calculated rate of fall of temperature equals or exceeds the set rate of temperature fall to be expected when said pacifier falls out of the mouth of said baby, such rate of temperature change causing said detection circuit to generate said first signal.

14. The baby pacifier as claimed in claim 1, wherein said detection circuit further includes means to detect temperature fall under a certain predetermined value.

15. The baby pacifier as claimed in claim 12, wherein the rate and/or the trend of temperature fall causing said detection circuit to generate said first signal is set in the range 0.5-10 degrees centigrade per minute.

16. The baby pacifier as claimed in claim 1, utilized by analog hardware, or digital hardware or software or a combination thereof.
